(19) United States
(12) Patent Application Publication
(10) Pub. No.: US 2016/0174879 A1
(43) Pub. Date: Jun. 23, 2016

(54) Smartphone Blink Monitor

(71) Applicant: Ziv Yekutieli, Zichron Yaakov (IL)

(72) Inventor: Ziv Yekutieli, Zichron Yaakov (IL)

(21) Appl. No.: 14/816,090

(22) Filed: Aug. 3, 2015

Related U.S. Application Data
(63) Continuation-in-part of application No. 14/578,364, filed on Dec. 20, 2014, now abandoned.

(51) Int. Cl. A61B 5/11; A61B 5/00; A61B 5/16

(52) U.S. Cl. CPC A61B 5/1128; A61B 5/1101; A61B 5/1107; A61B 5/165; A61B 5/4809; A61B 5/4082

(57) ABSTRACT

An application is employed on a smartphone which is adapted for vision processing of the user's face, in particular being adapted to detect when the user blinks. Other facial expressions may likewise be analyzed and detected. Occurrences of blinking and possibly various other facial expressions are logged, locally and/or to a remote server in communication with the application.

### CLAIM OF BENEFIT

This application claims benefit of U.S. patent application Ser. No. 14/578,364 filed 20 Dec. 2014, being a continuation in part thereof, which itself claims priority from U.S. provisional application 61/928,030 filed 16 Jan. 2014.

### FIELD OF THE INVENTION

The present invention relates to the field of diagnostic tests administered by means of smartphone.

### BACKGROUND OF THE INVENTION

Blinking, grimacing and other facial expressions and tics have been linked to various states, such as tiredness, mendacity, stress, and others. It is also linked to or may be an indicator of neurological disorders such as Parkinson's Disease, schizophrenia and others. Generally analysis of such expression is bound by the perspicacity of the subjects' everyday contacts, or is restricted to clinical settings.

There is thus a longfelt need for facial expression analysis by transparent, computerized means.

### SUMMARY OF THE INVENTION

It is within provision of the invention to use a smartphone to analyze facial gestures, especially blinking but also possibly including grimaces, tics, smiles, frowns, and any other expression or muscular contraction.

The application of the invention runs, for instance, in the background on a smartphone and therefore does not require or attract the users' attention. The application is adapted for vision processing of the user's face, in particular being adapted to detect when the user blinks. In some embodiments of the invention, other facial expressions are likewise analyzed and detected. Once detected, the occurrences of the various facial expression are logged, locally and/or to a remote server in communication with the inventive application.

The foregoing embodiments of the invention have been described and illustrated in conjunction with systems and methods thereof, which are meant to be merely illustrative, and not limiting. Furthermore just as every particular reference may embody particular methods/systems, yet not require such, ultimately such teaching is meant for all expressions notwithstanding the use of particular embodiments.

### DETAILED DESCRIPTION OF PREFERRED EMBODIMENTS

The present invention will be understood from the following detailed description of preferred embodiments, which are meant to be descriptive and not limiting. For the sake of brevity, some well-known features, methods, systems, procedures, components, circuits, and so on, are not described in detail.

The invention uses a smartphone or other computing platform with camera to analyze facial gestures, especially blinking but also possibly including grimaces, tics, smiles, frowns, and any other expression or muscular contraction.

The application of the invention runs, for instance, in the background on a smartphone and therefore does not require or attract the users' attention. The application is adapted for vision processing of the user's face, in particular being adapted to detect when the user blinks. In some embodiments of the invention, other facial expressions are likewise analyzed and detected. Once detected, the occurrences of the various facial expression are logged, locally and/or to a remote server in communication with the inventive application.

The occurrences may be logged in conjunction with other information such as time and contextual cues such as screencaptures of what the user was observing when blinking, and other contextual information. Statistical analyses may be undertaken based upon frequency of blinking, timing, duty cycle, and other factors. These may be found useful in determining correlations and/or predicting associated states such as tiredness, mendacity, and the like.

It is within provision of the invention to detect Parkinson's disease, schizophrenia, eye dystonia, blepharospasm, Brueghel's syndrome, oral facial dystonia, oromandibular dystonia, and the like. It is further within provision of the invention to correlate such occurrences of blinking with various underlying causes such as the conditions mentioned above as well as bright light, dust, smoke, foreign bodies in the eye, allergies, infections, dry eyes, stress, anxiety, fatigue, and congenital glaucoma. Some of these may be detected by means of image processing of the user's habitat and environment, which may provide information concerning the illumination/smokiness of the environment, clarity of the user's eyes, state of alertness of the user as determined by facial image analysis, and the like.

The foregoing description and illustrations of the embodiments of the invention has been presented for the purposes of illustration. It is not intended to be exhaustive or to limit the invention to the above description in any form.

Any term that has been defined above and used in the claims, should be interpreted according to this definition.

The reference numbers in the claims are not a part of the claims, but rather used for facilitating the reading thereof. These reference numbers should not be interpreted as limiting the claims in any form.

1. A diagnostic method for detection of the occurrence of blinking in a subject, consisting of the steps:
a. capturing a subject's facial features over a period of time by means of a camera;
b. analyzing said facial features to detect episodes of blinking by means of image analysis;
c. storing contextual information pertaining to said episodes.

2. The method of claim 1 wherein said analysis employs face detection to determine the position of a subject's face and eye detection to determine the position of said subject's eyes.

3. The method of claim 1 further determining information selected from the group consisting of:
the illumination/smokiness of the environment, clarity of the user's eyes, state of alertness of the user as determined by image analysis of the user's face, and facial expressions including smiles, frowns, grimaces.

4. The method of claim 1 wherein said camera is a smartphone camera and said analysis occurs by means of said smartphone.

5. The method of claim 2 wherein said contextual information is selected from the group consisting of: date, time, applications(s) running on on said smartphone.

6. The method of claim 2 wherein the frequency of blink occurrence is analyzed over time.

7. The method of claim 3 wherein the occurrence of said blink episodes and said contextual information and sent to a networked server.

8. The method of claim 3 using said information to assist in diagnoses.
